(19) United States
(12) Patent Application Publication
(10) Pub. No.: US 2011/0270097 A1
(43) Pub. Date: Nov. 3, 2011

(54) METHOD AND APPARATUS FOR ASSESSMENT OF FLUID RESPONSIVENESS

(76) Inventors: Pedro Mateo Riobo Aboy, Portland, OR (US); Cristina Crespo Veiga, Portland, OR (US)

(21) Appl. No.: 13/097,041

(22) Filed: Apr. 28, 2011

Related U.S. Application Data
(60) Provisional application No. 61/328,638, filed on Apr. 28, 2010.

(51) Int. Cl. A61B 5/0205; A61B 5/02

(52) U.S. Cl. 600/484; 600/481

(57) ABSTRACT

Disclosed embodiments include a method and related apparatus for determining a physiological parameter from the arterial blood pressure signal, the photoplethysmographic signal, the electrocardiogram, or any other a physiologic signal whose pulse amplitude variation is affected by respiration and it is indicative of fluid status in order to quantify the degree of amplitude modulation due to respiration (pulse variation) and help assess fluid responsiveness.

### CROSS-REFERENCE TO RELATED APPLICATIONS

This application claims the benefit of U.S. Provisional Application No. 61/328,638 filed on 2010-04-28 by the present inventors, which is incorporated herein by reference.

### TECHNICAL FIELD

Disclosed embodiments relate to methods and apparatus for physiological signal monitoring. Specifically, they relate to methods and apparatus for dynamic estimation of fluid responsiveness.

### BACKGROUND

Indicators and methods for noninvasive determination of fluid status of patients are important for monitoring the condition of critical care patients. In many critical care settings clinicians must decide whether patients should be given intravenous fluid boluses and other therapies to improve perfusion. Excessive fluid can be damaging by impairing lung function when it decreases oxygen delivery to tissues and contributes to organ failure. Insufficient fluid can result in insufficient tissue perfusion which can also contribute to organ failure. Determining the best course of fluid therapy for a given patient is difficult and clinicians have few clinical signs to guide them.

Fluid administration in hemodynamically unstable patients is often a major challenge when it comes to measuring stroke volume (SV), cardiac output (CO), or other hemodynamic parameters in real time. Correct clinical assessment of hypovolemia is difficult, as is the decision to undertake fluid resuscitation as the initial treatment strategy. Specifically, it is very difficult to predict whether a hemodynamically unstable patient will respond to fluid therapy with an increase in SV and CO. Moreover, fluid overload can cause significant pulmonary or cardiac dysfunction, whereas fluid insufficiency may cause tissue damage resulting in vital organ dysfunction. A patient's fluid responsiveness is the major and most important determinant to assess the adequacy of fluid resuscitation therapy and to ensure optimal cardiac performance and organ perfusion.

There are several dynamic parameters that can be used to assess fluid responsiveness from arterial blood pressure (ABP) and in some cases from plethysmogram signals. Several bedside indicators of ventricular preload have been used as predictors of fluid responsiveness. Right arterial pressure (RAP) and pulmonary artery occlusion pressure (PAOP) are commonly used in the intensive care unit (ICU) when deciding to administer fluids. Other bedside indicators of ventricular preload include right ventricular end diastolic volume (RVEDV) and left ventricular end diastolic area (LVEDA) measured with transesophageal echocardiography. Several studies and case reports have shown, however, that these static indicators based on cardiac filling pressures may have poor predictive value and often fail to give adequate information about fluid responsiveness.

Several studies have confirmed the clinical significance of monitoring the variations observed in left ventricular stroke volume that result from the interaction of the cardiovascular system and the lungs under mechanical ventilation. These stroke volume variations (SVV) are caused by the cyclic increases and decreases in the intrathoracic pressure due to the mechanical ventilation, which lead to variations in the cardiac preload and afterload. SVV has recently been extensively investigated and several studies have shown the usefulness of using SVV as predictor of fluid responsiveness in various clinical situations. Several other parameters based on SVV have been found to be useful as well. In particular, systolic pressure variation (SPV) with its delta-Up and delta-Down components has been found to be a very useful predictor of fluid responsiveness. SPV is based on the changes in the arterial pulse pressure due to respiration-induced variations in stroke volume. Yet another parameter that has been recently investigated and shown to be a valid indicator of fluid responsiveness is the pulse pressure variation (PPV).

The pulse pressure variation (PPV) index is a measure of the respiratory effect on the variation of systemic arterial blood pressure in patients receiving full mechanical ventilation. It is a dynamic predictor of increase in cardiac output due to an infusion of fluid. Numerous studies have demonstrated that PPV is one of the most sensitive and specific predictors of fluid responsiveness. Specifically, PPV has been shown to be useful as a dynamic indicator to guide fluid therapy in different patient populations receiving mechanical ventilation. For instance, PPV was found to exhibit better performance as a predictor of fluid responsiveness in patients before off-pump coronary artery bypass grafting than standard static preload indexes. PPV has also been shown to be useful for predicting and assessing the hemodynamic effects of volume expansion and a reliable predictor of fluid responsiveness in mechanically ventilated patients with acute circulatory failure related to sepsis. Another study concluded that PPV can be used to predict whether or not volume expansion will increase cardiac output in postoperative patients who have undergone coronary artery bypass grafting. A critical review of studies investigating predictive factors of fluid responsiveness in intensive care unit patients concluded that PPV and other dynamic parameters should be used preferentially to static parameters to predict fluid responsiveness.

The standard method for calculating PPV often requires simultaneous recording of arterial and airway pressure. Pulse pressure (PP) is manually calculated on a beat-to-beat basis as the difference between systolic and diastolic arterial pressure. Maximal PP (PPₘₐₓ) and minimal PP (PPₘᵢₙ) are calculated over a single respiratory cycle, which is determined from the airway pressure signal. Pulse pressure variations APP are calculated in terms of PPₘₐₓ and PPₘᵢₙ and expressed as a percentage,

$$PPV ( \% ) = 100 \times \frac{{PP}_{max} - {PP}_{min}}{( {PP}_{max} + {PP}_{min} ) / 2} \qquad ( 1 )$$

There are few available methods to automatically estimate PPV accurately and reliably. A limitation of current PPV methods is that they may not work adequately in regions of abrupt hemodynamic changes and this may limit their applicability in operating room environments.

### SUMMARY

Disclosed embodiments include a method and apparatus for assessing fluid responsiveness and guiding fluid therapy implemented in a medical system with one or more processors, comprising: (a) detecting and segmenting a physiological signal acquired by the medical system into a plurality of beats, each beat corresponding to a single cardiac cycle; (b) computing the upper and lower envelopes of the physiological signal from the plurality of beats; (c) computing an indicator substantially equivalent to a pulse amplitude variation based on the upper and lower envelopes; (d) filtering the indicator to remove signal artifacts present in the physiological signal resulting in a robust indicator of fluid responsiveness; and (e) displaying the robust indicator of fluid responsiveness, comparing it against one or more clinically relevant thresholds, and using it to assess fluid responsiveness and guide fluid therapy based on the robust indicator and one or more thresholds. According to one particular embodiment, and without limitation, the physiological signal can be an arterial blood pressure signal, a plethysmographic signal, an electrocardiogram signal, or any other cardiovascular signal correlated with pulse pressure variation changes. According to one particular embodiment, and without limitation, the robust indicator of fluid responsiveness is especially adapted for removing artifacts present in operating room and critical care environments.

### BRIEF DESCRIPTION OF THE DRAWINGS

Disclosed embodiments are illustrated by way of example, and not by way of limitation, in the figures of the accompanying drawings.

FIG. 1 shows a block diagram of the method according to one embodiment.

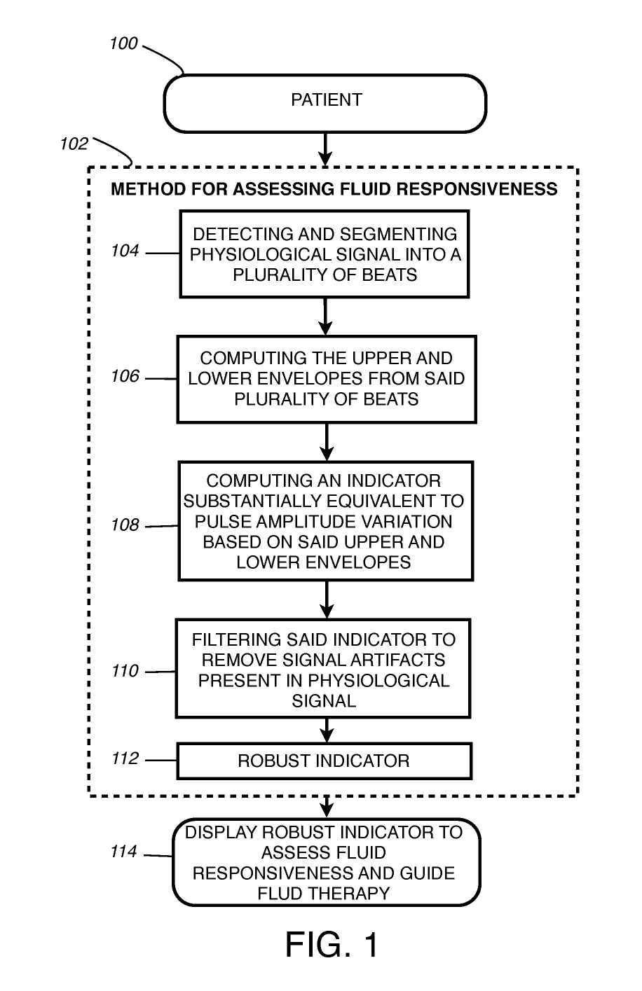

FIG. 2 shows the variation of pulse amplitude over two respiratory cycles.

FIG. 3 shows the detection of maxima and minima points on a physiological signal.

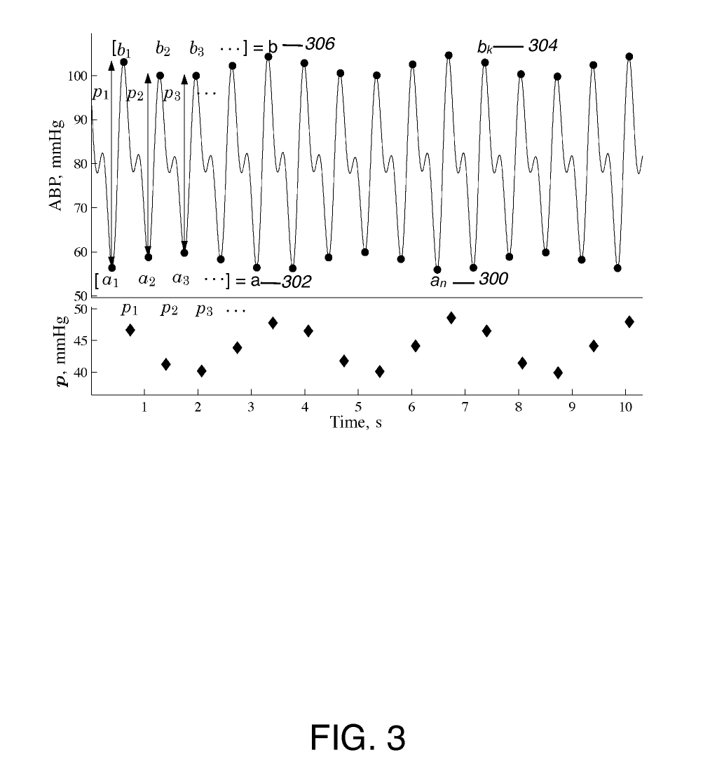

FIG. 4 shows continuous calculation of upper and lower envelopes and said indicator.

FIG. 5 shows the general block diagram of a medical apparatus that assesses fluid responsiveness and guides fluid therapy.

FIG. 6 shows a performance comparison of the proposed method to two other methods.

FIG. 7 shows a performance comparison of the proposed method to two other methods.

### DETAILED DESCRIPTION

Disclosed embodiments include a method for assessing fluid responsiveness and guiding fluid therapy 102 implemented in a medical system with one or more processors 502. According to one embodiment the method comprises the steps of (a) detecting and segmenting a physiological signal acquired by a medical system into a plurality of beats 104, each beat corresponding to a single cardiac cycle; (b) computing the upper and lower envelopes of the beat detected in the previous step 106; (c) computing an indicator substantially equivalent to a pulse amplitude variation based on the upper and lower envelopes of the physiological signal 108; (d) filtering this indicator to remove signal artifacts present in the physiological signal 110, resulting in a robust indicator of fluid responsiveness 112; and (e) ouputing (e.g., displaying) the resulting robust indicator of fluid responsiveness 114, comparing it against one or more clinically relevant thresholds, in order to assess fluid responsiveness and guide fluid therapy.

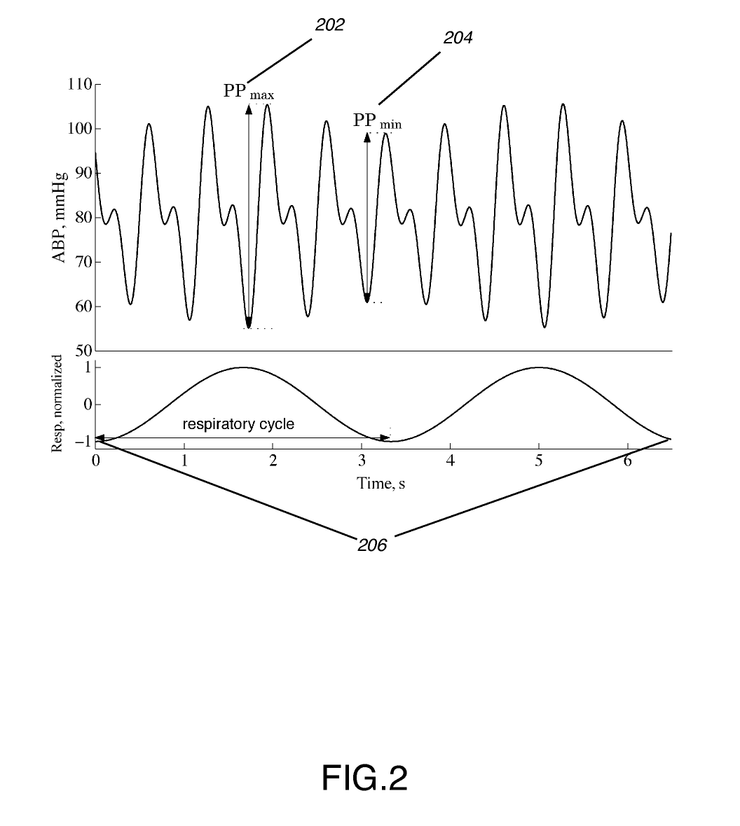

The method can be applied to any physiological signal whose pulse amplitude variation is affected by respiration and is indicative of fluid status. The method can therefore be applied to physiological signals such as arterial blood pressure signals, plethysmographic signals, and electrocardiogram signals. The pulse amplitude variation of any of these signals can be monitored continuously to indicate fluid status. FIG. 2 shows an example of the pulse amplitude variation of an arterial blood pressure signal over two respiratory cycles 206, 202 shows the maximum pulse amplitude variation and 204 shows the minimum pulse amplitude variation. Note that the pulse amplitude changes over the course of multiple respiratory cycles 206.

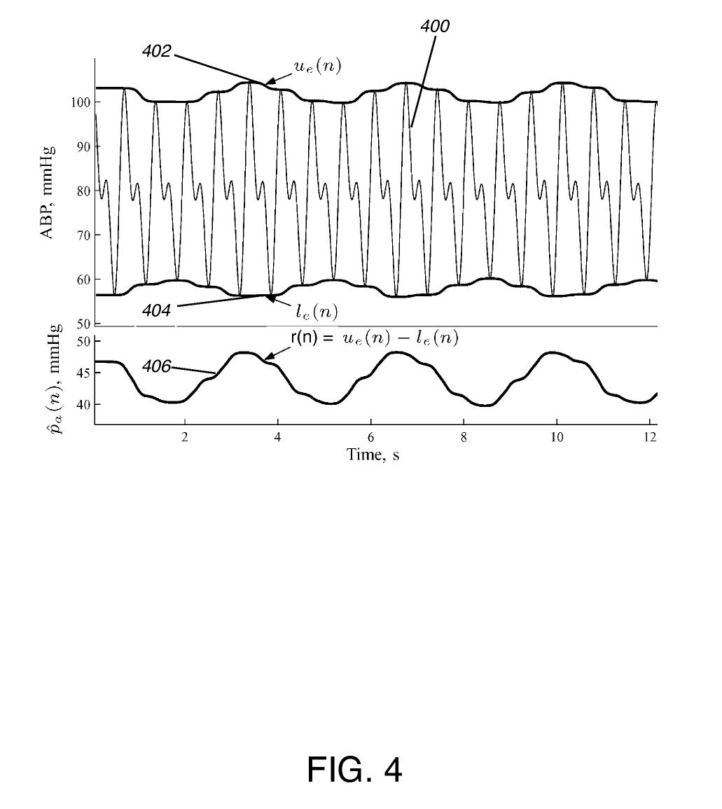

FIG. 4 shows that the pulse amplitude variation 406, an indicator of fluid status, can be estimated from the difference in the amplitudes of an upper 402 and lower 404 envelope of a physiological signal 400. According to a particular embodiment the upper 402 and lower 404 envelopes and indicator 406 are continuously calculated, that is, there is an estimate of the envelope at each sample.

Artifacts in the physiologic signal 608 can occur in operating room and critical care environments. Critical care environments can include any situation where immediate attention to a patient is needed, such as ambulatory care. A patient with a traumatic or life threatening injury may be undergoing first aid or surgery procedures. These procedures typically involve the movement of the patient 100, or manipulation of the patient's body. These movements and manipulations could cause artifacts to occur in a physiological signal acquisition system 504 that is in contact with the patient's body. According to a particular embodiment, filtering of said indicator 110 is used to remove artifacts introduced in critical care and operating room environments. The filtering of the indicator results in an indicator that is robust to artifacts introduced in these environments. Furthermore, this robust indicator 112 can be used to determine fluid status, and as an indicator of fluid responsiveness 114.

Additional artifacts in the indicator become present during abrupt hemodynamic changes. Abrupt hemodynamic changes can occur in a patient losing a large volume of blood 616. According to a particular embodiment, filtering of the indicator 110 is used to remove artifacts present in the indicator due to these abrupt hemodynamic changes. The filtering of the indicator results in an indicator that is robust to artifacts introduced by these conditions. Furthermore, this robust indicator 112 can be used to determine fluid status, and as an indicator of fluid responsiveness 114.

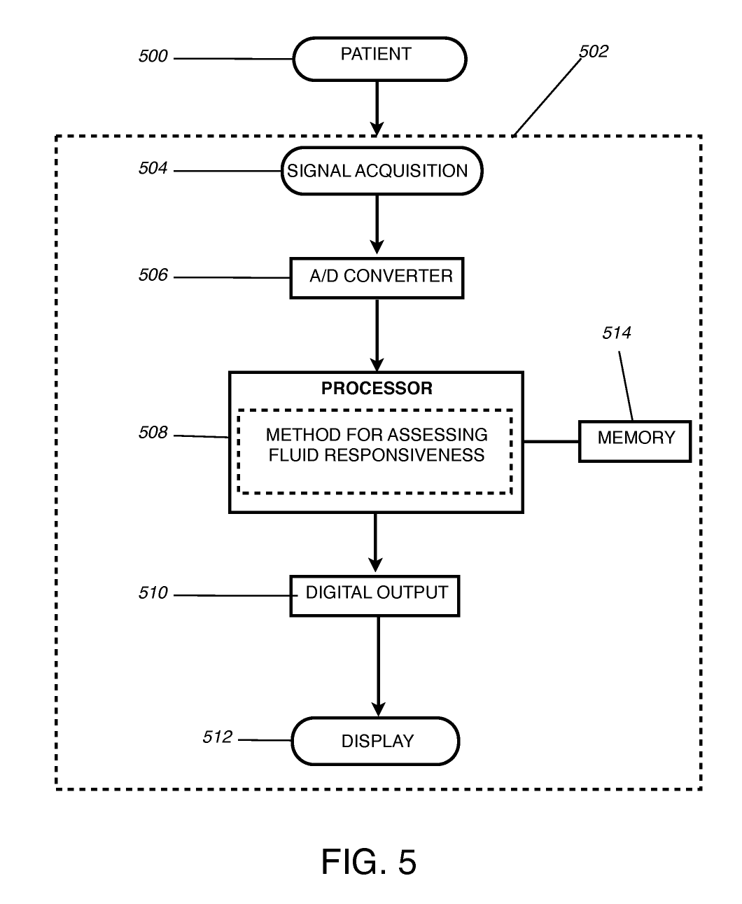

FIG. 5 shows the general block diagram of a medical apparatus that assesses fluid responsiveness and guides fluid therapy. According to a specific embodiment, the method can be implemented in a medical system with one or more processors 508, physiological signal acquisition 504, analog to digital converters 506, memory 514, and output devices 512 such as the typical bedside monitors used in clinical care (e.g., displays, printer for medical reports, etc). Alternatively, it can be implemented in a digital computer with one or more processors 508 to analyze physiological signals and display 512 or output 510, an indicator of fluid responsiveness that can be used to guide fluid therapy.

The following sections describe an embodiment of the method for assessing fluid responsiveness for the case of arterial blood pressure signals (APB) and estimation of the pulse pressure variation index (PPV). Substantially equivalent embodiments without departing from the spirit of the disclosed embodiments are applicable to other physiological signals and other derived parameters in addition to PPV. For instance, the method for assessing fluid responsiveness is applicable to the case of ABP, PLETH and ECG signals, as well as any other dynamic index predictive of fluid responsiveness substantially equivalent to a variation in pulse pressure of said physiological signal due to respiration.

### A. Method Description

### Step 1: Beat Detection and Segmentation 104

An automatic beat detection method for pressure signals is applied to the input pressure signal x(n) to identify the time instance corresponding to the beginning of each beat,

a=f(x(n)), (2)

where f(x(n)) denotes the operation of applying the detection method to the input signal x(n). The result of this operation is a vector a 302 that contains the sample indices corresponding to the beginning of each beat (i.e. the minima preceding each beat 300),

a=(a₁, a₂, . . . , a_{N}). (3)

Based on the vector a=(a₁, a₂, . . . , a_{N}) 302, the data is segmented as a set of N vectors corresponding to the N beats present in the signal,

$$\begin{matrix}{x}_{1} = ( x ( {a}_{1} ) , x ( {a}_{1} + 1 ) , … , x ( {a}_{2} - 1 ) ) \\ {x}_{2} = ( x ( {a}_{2} ) , x ( {a}_{2} + 1 ) , … , x ( {a}_{3} - 1 ) ) \\ ⋮ \\ {x}_{N} = ( x ( {a}_{N} ) , x ( {a}_{N} + 1 ) , … , x ( L ) ) ,\end{matrix} \qquad ( 4 )$$

where x(L) denotes the last sample in x(n). In practice, any automatic detection method with good performance can be used to perform this task.

### Step 2: Beat Maxima Detection

Given the set of vectors {xₖ}ₖ₌₁^{N} corresponding to the segmented signal x(n), the method detects the time index bₖ 304 corresponding the maximum in each segment,

$$\begin{matrix}{b}_{1} = arg {max}_{{a}_{1} < n < {a}_{2} - 1} {x}_{1} \\ {b}_{2} = arg {max}_{{a}_{1} < n < {a}_{3} - 1} {x}_{2} \\ ⋮ \\ {b}_{N} = arg {max}_{{a}_{N} < n < L} {x}_{N} .\end{matrix} \qquad ( 5 )$$

The result of this step is a vector b 306 that contains the sample indices corresponding to the maxima of each beat,

$$b = arg {max}_{{a}_{i} < n < {a}_{i + 1} - 1} {\{ x \}}_{k = 1}^{N} = ( {b}_{1} , {b}_{2} , … , {b}_{N} ) . \qquad ( 6 )$$

### Step 3: Envelope Estimation

In the next step the method estimates the upper uₑ(n) 402 and lower lₑ(n) 404 envelopes from the x(b) and x(a) time series, respectively. This is accomplished by smoothing and uniformly resampling x(a) and x(b) at a rate of fₛ with a kernel smoother,

$$\begin{matrix}{u}_{e} ( n ) = \frac{{\sum }_{k = 1}^{N} x ( b ) ) b ( \frac{\vert {nT}_{s} - t ( k ) \vert}{{σ}_{b}} )}{{\sum }_{k = 1}^{N} b ( \frac{\vert {nT}_{s} - t ( k ) \vert}{{σ}_{b}} )} & ( 7 ) \\ {l}_{e} ( n ) = \frac{{\sum }_{k = 1}^{N} x ( a ) ) b ( \frac{\vert {nT}_{s} - t ( k ) \vert}{{σ}_{b}} )}{{\sum }_{k = 1}^{N} b ( \frac{\vert {nT}_{s} - t ( k ) \vert}{{σ}_{b}} )} & ( 8 )\end{matrix}$$

where

$${T}_{s} = \frac{1}{{f}_{s}}$$

is the resampling interval with fₛ corresponding to the original sampling frequency of x(n), t(k) denotes the times of the signal observations, σ_{b} is the kernel width, and b(·) is a clipped Gaussian kernel function,

$$b ( u ) = \{ \begin{matrix}exp ( \frac{- {u}^{2}}{2} ) & if - 5 \leq u \leq 5 \\ 0 & otherwise .\end{matrix} \qquad ( 9 )$$

The kernel width controls the degree of smoothing and depends on the fundamental frequency of the pressure signal (heart rate). A width of 0.2 s works well for heart rates up to 4 Hz.

### Step 4: Pulse Pressure Variation Estimation

The method uses the estimated upper uₑ(n) 402 and lower lₑ(n) 404 envelopes to obtain a continuous estimate of the pulse pressure 406,

r(n)=u_{c}(n)−l_{c}(n) (10)

the estimate of pulse pressure r(n) also serves as an estimate of the respiratory signal during mechanical ventilation. A pulse pressure data matrix R=(r₁, r₂, . . . , r_{M}) is created by partitioning the pulse pressure signal r(n) into M 50% overlapping vectors of dimension

$$D = 2 \frac{1}{{f}_{r}} {f}_{s} \times 1 ,$$

$$M = 2 {T}_{r} \frac{2 L}{{f}_{s}} \qquad ( 11 )$$

where

$${T}_{r} = \frac{1}{{f}_{r}}$$

is the average respiratory period and fᵣ the respiratory frequency, L denotes the number of samples in x(n), uₑ(n), lₑ(n) and r(n), and fₛ is the sampling frequency. Given the set of vectors {rₖ}ₖ₌₁^{M}, the method detects the time index cₖ corresponding the minimum in each segment,

$$\begin{matrix}{c}_{1} = arg {min}_{1 < n < D} {r}_{1} \\ {c}_{2} = arg {min}_{1 < n < D} {r}_{2} \\ ⋮ \\ {c}_{M} = arg {min}_{1 < n < D} {r}_{M} .\end{matrix} \qquad ( 12 )$$

The result of this step is a vector c that contains the sample indices corresponding to the minima of each r,

$$c = arg {min}_{1 < n < D} {\{ r \}}_{k = 1}^{M} = ( {c}_{1} , {c}_{2} , … , {c}_{M} ) . \qquad ( 13 )$$

Analogously, the method detects the time index dₖ corresponding the maximum for each vector r,

$$d = arg {max}_{1 < n < D} {\{ r \}}_{k = 1}^{M} = ( {d}_{1} , {d}_{2} , … , {d}_{M} ) . \qquad ( 14 )$$

The raw pulse pressure variation index y is obtained from the c and d vectors,

$$y = 2 \frac{x ( d ) - x ( c )}{x ( d ) + x ( c )} = 2 m \qquad ( 15 )$$

where m denotes the amplitude modulation (AM) index defined for large carrier double side-band AM. The y vector may contain erroneous values in regions where the input pressure signal x(n) is corrupted by artifact. Thus, the final estimate of the pulse pressure variation index \hat{p} is obtained by applying a recursive filter to process the raw measurements y,

\hat{p}_{n+1|n+1}=\hat{p}_{n+1|n}+Kₙ₊₁(yₙ₊₁−\hat{p}_{n+1|n}) (16)

Domain knowledge about the evolution of the true pulse pressure variation p is incorporated into the estimator by constraining the pulse pressure variation index to evolve slowly,

pₙ₊₁=pₙ+uₙ (17)

that is, the pulse pressure variation index at time n+1, pₙ₊₁, is equal to the pressure variation index at the previous time n, pₙ, within some error uₙ.

The gain Kₙ₊₁ is a function of the difference between the measured and the estimated pulse pressure variation index based on the model, eₙ₊₁=yₙ₊₁−\hat{p}_{n+1|n},

$${K}_{n + 1} = \{ \begin{matrix}{κ}_{1} & if \vert {e}_{n + 1} \vert = \vert {y}_{n + 1} - {\hat{p}}_{n + 1 \vert n} \vert \leq {ξ}_{1} \\ {κ}_{2} & if {ξ}_{1} \leq \vert {e}_{n + 1} \vert = \vert {y}_{n + 1} - {\hat{p}}_{n + 1 \vert n} \vert \leq {ξ}_{2} \\ {κ}_{3} & if \vert {e}_{n + 1} \vert = \vert {y}_{n + 1} - {\hat{p}}_{n + 1 \vert n} \vert \geq {ξ}_{2}\end{matrix} \qquad ( 18 )$$

where the vectors K=(κᵢ, κ₂, κ₃) and T=(ξ₁, ξ₂) are user-specified parameters. By default the method implementation uses K=(1, 0.5, 0) and T=(1, 25). For these specific K and T, the method discards pulse pressure variation measurements y when the residual |eₙ₊₁|=|yₙ₊₁−\hat{p}_{n+1|n}|≧ε₂, since the pulse pressure variation is not supposed to change so significantly from one respiratory cycle to the next. Analogously, the method updates the predicted pulse pressure variation based on the model using the measurements y when |eₙ₊₁=|yₙ₊₁−\hat{p}_{n+1|n}|≦ε₁, since this variability can be considered physiological in nature. For values of the residual ε₁≦|eₙ₊₁|≦ε₂, the predicted pulse pressure variation based on the model is \hat{p}_{n+1|n} is updated by κ₂eₙ₊₁=0.5eₙ₊₁. Note that this recursive filter implements a Kalman filter where the state pₙ₊₁=pₙ+uₙ is modeled as a slowly changing process, and the noisy measurements are linearly related to the state yₙ₊₁=pₙ+vₙ. However, the Kalman gains Kₙ₊₁ are not computed based on the error covariance matrix and are only approximate. Thus, the piecewise constant gains K=(κ₁, κ₂, κ₃) are suboptimal.

### B. Method Assessment

The method was compared against an existing PPV monitoring method and validated prospectively against expert annotated arterial blood pressure (ABP) signals. The method was developed using pressure signals from different subjects than those used for performance assessment. The assessment was measured only once without any parameter tuning. It is important to note that the underlying method has already been thoroughly assessed as part of a clinical study by Cannesson. Thus, the objective is not to conduct a clinical assessment regarding fluid responsiveness or to produce Bland-Altman plots of the method against manually annotated PPV. The objective is to present the performance of the method compared to an existing commercial method during regions of abrupt hemodynamic changes. The database used for our study was composed of 18 ABP signals sampled at 50 Hz obtained from 18 mechanically ventilated crossbred Yorkshire swine (over 40 hours of ABP recordings). These recordings were acquired at the Animal Laboratory of the Oregon Health and Science University (Portland, Oreg., USA). The subjects underwent Grade V liver injury after splenectomy, while receiving mechanical ventilation, and general anesthesia with isoflurane. All subjects in the database underwent a period of abrupt hemodynamic change after an induced Grade V liver injury involving severe blood loss resulting in hemorrhagic shock, followed by fluid resuscitation with either 0.9% normal saline or lactated ringers solutions. Trained experts manually calculated PPV at five time instances during the period of abrupt hemodynamic changes. These expert manual annotations provide a “gold-standard”for method comparison and validation. The study protocol was reviewed and approved by the Institutional Review Board at Oregon Health and Science University.

### C. Example Results

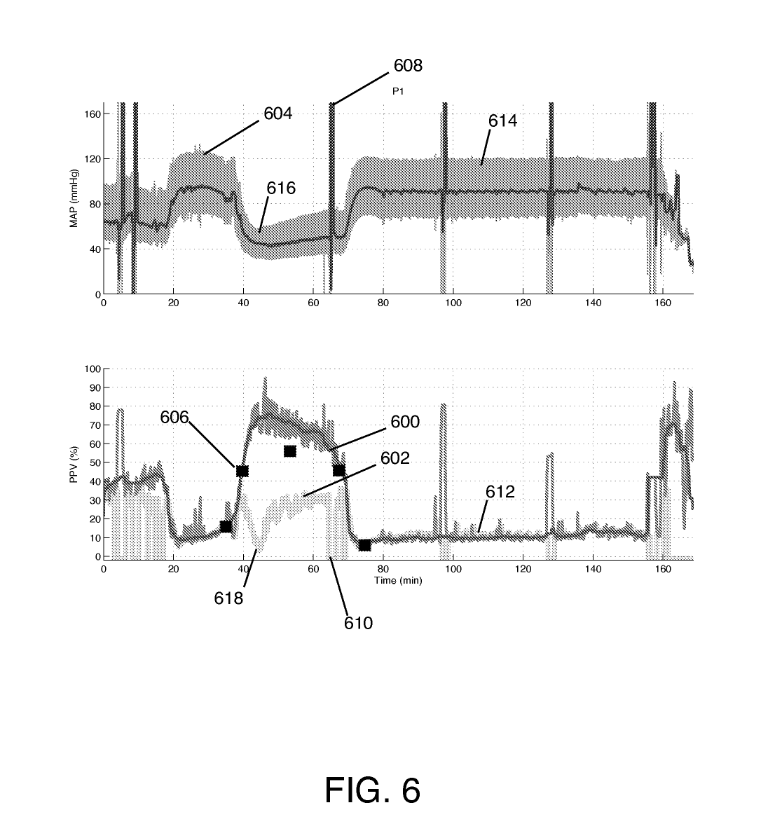

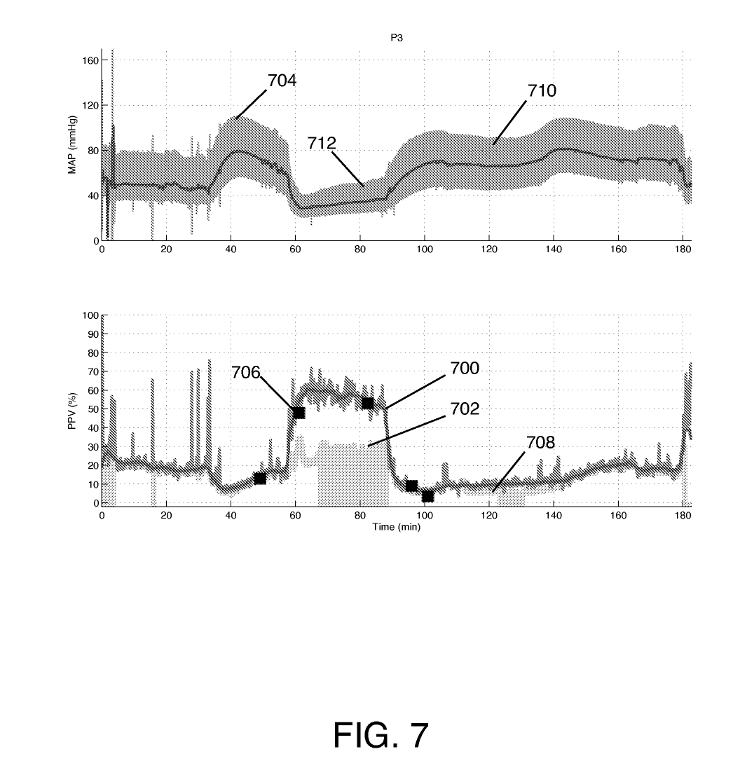

FIG. 6 shows a comparison of the proposed enhanced PPV method (dark grey) 600 against a commercial PPV monitoring method (light grey) 602 for the one of the first nine subjects. FIG. 7 shows a comparison of the proposed enhanced PPV method (dark grey) 700 against a commercial PPV monitoring method (light grey) 702 for another one of the first nine subjects. For each subject the top plot shows the ABP signal 604, 704 and the bottom plot shows the estimated PPV using both methods 600, 602, 700, 702. Five “gold-standard” PPV manual annotations calculated by trained experts during periods of abrupt ABP changes are shown as black squares on the bottom plot 606, 706 of each subject. Note that both methods are consistent 612, 708 for the most part during periods where the ABP signal is relatively stationary 614, 710. The proposed method has better performance than the commercial PPV monitoring method during periods of abrupt ABPM changes. FIG. 6 shows an examples where the ABP signal was severely corrupted by artifact 608. In this case the commercial PPV method fails to provide an adequate PPV value 610. Note, however, how the proposed method 600 is robust to these types of artifact 608. In these regions the method discards the pulse pressure variation measurements y because the residual |eₙ₊₁|=|yₙ₊₁−\hat{p}_{n+1|n}|≧ε₂. Since the pulse pressure variation is not supposed to change so significantly from one respiratory cycle to the next, the method performs a time update (model-based prediction) and no measurement update.

The commercial PPV monitoring method 612, 708 performed well in regions of normal hemodynamic changes 614, 710. However, the commercial PPV monitoring method 618, 702 failed to accurately estimate the PPV during the periods between the injury and fluid resuscitation 616, 712 in both of these subjects, and consequently it failed to predict fluid responsiveness during the periods of severe blood loss. These results indicate that while the commercial PPV monitoring method is a useful tool to estimate PPV and predict fluid responsiveness in situations where normal hemodynamic changes are expected, it may not provide accurate PPV values during abrupt hemodynamic changes.

As shown in FIG. 6 and FIG. 7, the proposed method is capable of accurately estimating the PPV index during periods of significant hemodynamic changes 616, 712 and is robust to artifact 608, 600. Note that in all the subjects the PPV estimates obtained with this method are consistent with the PPV expert annotations 606, 706. The overall absolute error over all subjects is 2083.19 for the commercial PPV monitoring method, nearly four times greater than the error of 555.18 from the proposed method.

Our results show that the proposed method to estimate PPV is an improvement over currently available methods. The commercial PPV monitoring method has already been thoroughly validated in a clinical study and has been found to be both accurate and useful in clinical environments. However, there is a need to improve upon this method currently implemented as part of a commercial system, particularly to make it more robust to artifact and accurate during regions of abrupt hemodynamic changes.

While particular embodiments have been described, it is understood that, after learning the teachings contained in this disclosure, modifications and generalizations will be apparent to those skilled in the art without departing from the spirit of the disclosed embodiments. It is noted that the foregoing embodiments and examples have been provided merely for the purpose of explanation and are in no way to be construed as limiting. While the method and corresponding medical apparatus have been described with reference to various embodiments, it is understood that the words which have been used herein are words of description and illustration, rather than words of limitation. Further, although the embodiments have been described herein with reference to particular means, materials and embodiments, the actual embodiments are not intended to be limited to the particulars disclosed herein; rather, they extend to all functionally equivalent structures, methods and uses, such as are within the scope of the appended claims. Those skilled in the art, having the benefit of the teachings of this specification, may effect numerous modifications thereto and changes may be made without departing from the scope and spirit of the disclosed embodiments in its aspects.

The invention claimed is:

1. A method for assessing fluid responsiveness and guiding fluid therapy implemented in a medical system with one or more processors, comprising:
(a) detecting and segmenting a physiologic signal acquired by said medical system into a plurality of beats, each beat corresponding to a single cardiac cycle;
(b) computing an upper and lower envelope of said physiological signal from said plurality of beats;
(c) computing an indicator substantially equivalent to a pulse amplitude variation based on said upper and lower envelope;
(d) filtering said indicator to remove signal artifacts present in said physiological signal resulting in a robust indicator of fluid responsiveness; and
(e) outputting said robust indicator of fluid responsiveness;
whereby said robust indicator of fluid responsiveness can be used to assess fluid responsiveness and guide fluid therapy by comparing it against one or more clinically relevant thresholds.

2. The method of claim 1, wherein said physiologic signal is any physiological signal whose pulse amplitude variation is affected by respiration and it is indicative of fluid status.

3. The method of claim 2, wherein said physiologic signal is an arterial blood pressure signal.

4. The method of claim 2, wherein said physiologic signal is a plethysmographic signal.

5. The method of claim 2, wherein said physiologic signal is an electrocardiogram signal.

6. The method of claim 2, wherein said upper and lower envelope and said indicator substantially equivalent to a pulse amplitude variation are continuously calculated.

7. The method of claim 6, wherein filtering said indicator to remove signal artifacts present in said physiological signal resulting in a robust indicator of fluid responsiveness is especially adapted for removing artifacts present in operating room and critical care environments.

8. The method of claim 7, wherein said filtering is especially adapted for removing artifacts present in operating room environments and artifacts due to abrupt hemodynamic changes.

9. The method of claim 8, wherein said filtering is based on a recursive filter that incorporates domain knowledge regarding the expected pulse pressure variation changes.

10. A medical apparatus for assessment of fluid responsiveness, said apparatus comprising:
(a) a signal acquisition circuit to acquire a physiologic signal whose pulse amplitude variation is affected by respiration and it is indicative of fluid status;
(b) a memory to store said physiologic signal;
(c) a processor configured to perform the processing step of (1) detecting and segmenting said physiologic signal acquired into a plurality of beats, each beat corresponding to a single cardiac cycle; (2) computing an upper and lower envelope of said physiological signal from said plurality of beats; (3) computing an indicator substantially equivalent to a pulse amplitude variation based on said upper and lower envelope; and (4) filtering said indicator to remove signal artifacts present in said physiological signal resulting in a robust indicator of fluid responsiveness; and
(d) an output device for outputting said dynamic indicator for assessment of fluid responsiveness.

11. The medical apparatus of claim 10, wherein said filtering is based on a recursive filter that incorporates domain knowledge regarding the expected pulse pressure variation changes.
